(19) United States
(12) Patent Application Publication
(10) Pub. No.: US 2004/0241784 A1
(43) Pub. Date: Dec. 2, 2004

(54) Method of selecting non-pathogenic gram-positive lactic acid bacteria strains

(76) Inventor: Pascal Butty, Fronsac (FR)

Correspondence Address: JACOBSON HOLMAN PLLC, 400 SEVENTH STREET N.W., SUITE 600, WASHINGTON, DC, 20004, US

(21) Appl. No.: 10/480,472

(22) PCT Filed: Jun. 14, 2002
(86) PCT No.: PCT/FR02/02058

(30) Foreign Application Priority Data
Jun. 14, 2001 (FR) 0107812
Oct. 29, 2001 (FR) 0113983

(51) Int. Cl. C12Q 1/04; C12N 1/20

(52) U.S. Cl. 435/34; 435/252.9

(57) ABSTRACT

The invention relates to a method of selecting non-pathogenic Gram-positive lactic acid bacteria strains that can combat infections by pathogenic bacteria for which the pathogenicity is the result of a tissue adhesion. The inventive method consists in choosing a strain presenting all of the following properties: a) the ability to adhere to tissues of an organ that is likely to be infected; b) the ability of the bacteria of said strain to fix to one another via mutual adhesion sites; and c) the ability to fix to the attachment determinants of the pathogenic bacterial strain responsible for the infection. The bacterial strains thus selected can be used in the production of therapeutic compositions that are intended to prevent or treat pathological conditions associated with the infection of the host's organ by pathogenic bacterial strains.

The invention relates to a method of selecting Gram-positive lactic acid bacteria strains which allows infections of a pathogenic nature by Gram-negative or Gram-positive bacteria strains, such as strains of enteropathogenic bacteria, to be combated.

Severe digestive pathologies induced by enteropathogenic bacteria are common in farm animals. This is true in particular of colibacillosis, which remains the main cause of diarrhoea encountered in pig farms. In the majority of cases, enteropathogenic bacterial strains of the Escherichia coli type are responsible for the diarrhoea.

The pathogenicity of strains of the Escherichia coli type is the result of tissue adhesion to the wall of the duodenum.

More generally, in the case of bacteria of which the pathogenicity is mediated by tissue adhesion, the adhesion is often stereospecific and can only take place if the tissue carries a very particular type of receptor. The interaction between a bacterial lectin and a tissue sugar is a typical example of stereospecific interaction. These lectin molecules are often carried by filamentous appendages called fimbriae.

Fimbriae are very fine protein filaments found mainly, and very commonly, in Gram-negative bacteria. They can be dispersed over the entire surface of the cell or be more localised. A single fimbria consists of repeated linear protein sub-units. These sub-units are often rich in non-polar amino acids, so that fimbriae-carrying cells tend to have more hydrophobic surfaces than those of cells without fimbriae. In Gram-negative bacteria, numerous types of fimbriae provide the adhesion of cells to one another or of cells to a particular surface. Each type of fimbria has its lectin and adheres only to a specific receptor consisting of glycoprotein and/or glycolipid epitopes.

Generally, enteropathogenic bacteria do not express their fixing appendages, such as fimbriae, in the environment, but only once ingested, the synthesis of these appendages being promoted by the physicochemical conditions of the digestive tract.

K88 Escherichia coli bacteria, which express K88 fimbriae as their fixing appendage, belong to this type of pathogenic bacteria.

K88 Escherichia coli colonises the duodenum, which does not contain as complex a protective flora as the other distal regions of the digestive tract, without difficulty.

The pathogen approaches the mucous membrane of the duodenum and fixes to the first receptors that are present in the mucus by chemotaxis. The pathogen then passes through the mucus gel to reach the epithelium of the subjacent duodenal villi, to which it fixes with avidity. It is the fixing of the pathogen which induces a series of intracellular mechanisms, resulting in the expression of a virulence.

The fixing of the K88 fimbriae on the receptors of the microvilli induces, in a first stage, the synthesis of enterotoxins by the pathogen, which causes, in a second stage, an inversion of the ionic channels and a loss of Cl⁻ ions associated with a permanent secretion of water by the enterocytes. In a mass infection, this phenomenon, taken as a whole, causes acute dehydration, often resulting in the death of the animal within a matter of hours.

In pigs, infections by K88 serotype bacterial strains are common in animals in pre-weaning and up to 2 weeks in post-weaning.

As far as animals aged between 40 and 75 days, i.e. in the second phase of post-weaning and up to the start of fattening, are concerned, infections are generally caused by 0138, K8 or K85 serotype bacterial strains.

Conventionally, bacterial infections are combated by administering antibiotics. The antibiotics are effective only in increasingly high doses, which has the drawback of multiplying the negative side-effects that result from excessive antibiotic therapy (Bartlett J. G., Clinical of Infectious Diseases, 1992, 15 (4), 573-581). This phenomenon is, however, inevitable in so far as the pathogenic bacteria, upon repeated contact with the antibiotics, tend to develop a resistance to antibiotics, which is responsible for the ineffectiveness of the treatments.

Other alternatives to antibiotic treatment have been proposed:

a first solution consists in using natural prebiotic compounds, added to food, that can selectively boost the growth of certain bacterial species that do not harm the intestinal flora, in order that the intestinal flora performs an optimal protective function in a minimum of time;

a second solution is based on the in vitro production of fecal flora or of cultures of bacterial strains, with the object of administering harmless exogenous bacteria, which serve to strengthen the constituent elements of the flora against undesirable pathogens, to animals via feed or drinking water.

This solution seems to have a certain effectiveness. Unfortunately, complex flora have to be handled and preserved extremely carefully if a constant optimal protection level is to be maintained.

A third solution consists in using a much more limited number of bacterial strains, isolated from a flora. The stages of selection of the most advantageous bacteria, as well as knowledge of their evolution in the digestive tract, have often been neglected.

All of these methods are unsatisfactory in so far as the strains obtained have often proven to be insufficiently effective in vivo.

The invention aims to improve the means of combating bacterial strains by providing a method of selecting probiotic bacteria strains that are particularly effective in physiological conditions.

More precisely, the invention relates to a method of selecting non-pathogenic Gram-positive lactic acid bacteria strains that can combat infections by pathogenic bacteria for which the pathogenicity is mediated by tissue adhesion, consisting in selecting a strain presenting all of the following properties:

a—the ability to adhere to tissues of an organ that is likely to be infected;

b—the ability of the bacteria of said strain to fix to one another via mutual adhesion sites; and

c—the ability to fix to the attachment determinants of the pathogenic bacterial strain responsible for the infection.

Preferably, the Gram-positive lactic acid bacteria strains are selected from the bacteria that are naturally present in the infected organ of the host.

The bacterial strains of which the pathogenicity is the result of tissue adhesion include the enteropathogenic strains of Escherichia coli. One of the most widespread is the strain identified by its ECET/0147: K88ac serotype (Erickson et al., Infection and Immunity, 1992, 60, 983-988).

Other examples of pathogenic enterobacteria of which the pathogenicity is the result of tissue adhesion include the Salmonellas. In these bacteria, which are more particularly responsible for infections encountered in poultry, the fimbriae are essential in initiating colonisation, in particular in the caecum of poultry, a stage that is preliminary to the infection.

A further example of a bacterial strain of which the pathogenicity is the result of tissue adhesion is Helicobacter pylori, which is responsible for chronic gastric inflammatory diseases and gastric and duodenal ulcers. The adhesion of Helicobacter to gastric cells by means of adhesins on the surface of the pathogen fixing to a receptor on the gastric mucous membrane is essential for initiating the ulceration process.

In the case of the pathogen Campylobacter jejuni, it is the liposaccharides which fulfil the adhesin function, enabling fixing to the epithelial cells and to the mucus, causing a severe diarrheic state in humans.

Yersinia or the Clostridies are other examples of species of bacterial strains of which the pathogenicity is the result of tissue adhesion.

The method of the invention is more particularly advantageous for selecting bacteria that can combat pathogenic bacteria expressing fimbriae with a view to stereospecific tissue adhesion.

The triple particularity of adherence which characterises the Gram-positive lactic acid bacteria selected by the method according to the invention provides improved antimicrobic activity.

This activity can be specific or non-specific.

It is preferred that the lactic acid bacteria selected also have the particularity of being able to fix in a selective manner to the attachment determinants of the pathogenic bacterial strain expressing fimbriae.

The selectivity can be displayed by demonstrating the absence of adhesion of the lactic acid bacteria strain to bacteria which do not express fimbriae and result from the mutation of the pathogenic bacterial strain expressing fimbriae.

Properties a), b) and c) of the lactic acid bacteria can be displayed in an independent manner.

Nevertheless, according to a preferred embodiment of the invention, properties b) and c) are displayed in a simultaneous manner in an appropriate buffered physiological medium. The physiological medium which can be used to do this must reflect the physiological conditions of the medium in which the lactic acid bacterium will ultimately have to exert its activity. The composition of the physiological medium may be determined by a person skilled in the art, in the light of his knowledge of the prior art as a function of the infected organ portion.

Generally, the buffered physiological medium will substantially contain various electrolytes influencing the pH, bile secretions, pancreatic secretions, gastric secretions and mucus extracts.

Preferably, the infected organ is a portion of the digestive tract.

If the infected organ is the duodenum, the physiological medium contains, in particular, a duodenal mucus extract and the enzymes of digestive secretions and, more particularly, of bile salts, gastric secretions and pancreatic digestive secretions.

Furthermore, chloride ions, in the form of salt and/or hydrochloric acid, may advantageously be introduced into the relevant physiological medium, the chloride ions being secreted naturally and present in the alimentary bolus of the pyloric region.

If the chloride ions are added in the form of hydrochloric acid, and as a function of the amount of hydrochloric acid introduced, it may be necessary to neutralise the acidity of the medium by adding a base, so as to restore the pH to the physiological pH, which is approximately 6 in the region of the mucous membrane of the middle duodenum.

The salts which can be used to incorporate chloride ions are preferably alkali or alkaline-earth metal salts, sodium chloride being preferred.

The use of inorganic bases, such as NaHCO₃, Na₂CO₃, KHCO₃ and K₂CO₃, as a base which can be used to restore the pH, is preferred; better still, NaHCO₃ and KHCO₃ are used.

In order to combat the bacterial infections which affect the porcine duodenum, for example, a physiological buffer having a pH between 5.5 and 6.5, preferably between 5.8 and 6.2, for example 6, based on porcine gastric pepsin, porcine gastric mucus, porcine pancreatin, porcine bile extract and an NaCl solution will be used.

Preferably, the physiological buffer contains:

| | |
|---|---|
| porcine gastric pepsin: | 2 g |
| porcine gastric mucus: | 1 g |
| porcine pancreatin: | 1 g |
| porcine bile extract: | 4.5 g |
| aqueous NaCl solution at 5 g/l: | sufficient for 1 litre |

Porcine gastric pepsin, porcine gastric mucus, porcine pancreatin and porcine bile extract are readily available on the market.

As far as the tissue adhesion properties in the infected organ (properties a) are concerned, they will easily be demonstrated in vitro by a person skilled in the art using conventional methods.

The adherence capacities of lactobacilli on the porcine duodenal wall are indicated by way of illustration:

in a buffer having a pH between 7 and 8, such as the Krebs-Henseleit buffer with the formulation:

| | |
|---|---|
| 0.12 M aqueous solution of NaCl: | 7 g |
| 0.014 M aqueous solution of KCl: | 0.1 g |
| 0.025 M aqueous solution of NaHCO₃: | 2.1 g |
| 0.001 M aqueous solution of KH₂PO₄: | 0.13 g |
| water: | sufficient for 1 litre, |
| | of which the pH is 7.4. |

According to a preferred embodiment of the selection method, properties b) and c) are thus assessed simultaneously in a buffered physiological medium reflecting the physiological conditions of the infected organ.

If the infected organ is the duodenum, the buffered physiological medium comprises a duodenal mucus extract, the enzymes of digestive secretions and, more particularly, of bile salts, gastric secretions and pancreatic digestive secretions, as well as the electrolytes that are naturally present in the duodenum and influence the pH.

The invention also relates to the use of Gram-positive lactic acid bacteria strains, selected according to the method of the invention, to produce a therapeutic composition that is intended to prevent or treat pathological conditions associated with an infection of the host organ by pathogenic bacterial strains.

According to a preferred embodiment of the invention, the Gram-positive lactic acid bacteria strain is naturally present in the infected host organ.

In an advantageous manner, the pathogenic bacterial strain is an enterotoxigenic or verotoxigenic strain and, for example, a strain of the species Escherichia coli.

According to the invention, the host is preferably an animal, such as a fowl, a pig, a bovine, a dog, a cat, a horse, an ovine, a caprine or a fish, preferably a pig.

The method of the invention, which aims to combat the bacterial infections affecting the porcine duodenum, will be illustrated hereinafter in more detail.

### EXAMPLE

The lactobacilli strains tested were strains newly isolated from the duodenum of piglets aged between 6 and 16 weeks or of sows aged 2 years. A single strain was isolated per animal. The animals were selected from various European farms. The strains were all isolated from an initial culture on MRS agar at 37° C. by micro-aerobiosis and were then identified according to a fermentation study of sugars on the API 50CH gallery. The main bacterial types that were finally tested were Lactobacillus and Leuconostoc.

The MRS (de Man, Rogosa and Sharpe) agar was prepared by dissolving 70 g of the following mixture A in 1 litre of water:

Mixture A:

| | |
|---|---|
| Peptone | 10 g |
| Cattle meat extract | 10 g |
| Yeast extract | 5 g |
| Dextrose | 20 g |
| Monoleate sorbitan complex | 1 g |
| Ammonium citrate | 2 g |
| Sodium acetate | 5 g |
| Magnesium sulphate | 0.1 g |
| Manganese sulphate | 0.05 g |
| Dibasic potassium phosphate | 2 g |
| Agar | 15 g |

The final pH of the agar was 6.5±0.2 at 25° C.

Cultures of these lactobacilli were prepared simply by withdrawing bacteria at the surface of the MRS agar and suspending them in the Krebs-Henseleit buffer to attain a value of 10⁹ bacteria/ml.

The pathogenic bacteria strain which it is desired to eradicate belongs to the species Escherichia coli. This strain was isolated in the digestive tract of a very sick animal. The severe diarrheic colibacillosis which had spread throughout an entire farm was caused by this ETEC LT+, 0149; K91, K88ac strain.

The lactobacilli were screened using the following three test procedures.

Test Procedure for Displaying Tissue Adherence Properties on the Mucous Membrane and on the Enterocytes of the Duodenum

A plurality of piglets aged 6 months were slaughtered and the duodena of these animals were cut up into 10 cm segments, which were vigorously washed with the Krebs buffer (buffer 1), defined above, so as to remove the intestinal chyme, mucus and residual bacteria debris. Once washed, the intestinal segments were opened over their entire length, then immersed in a glycerolised buffer (buffer 2, defined below) containing enzyme inhibitors, in order to achieve the structural stability of the mucous membrane during preservation. The samples were thus preserved at −20° C. The glycerol concentration was adjusted, in order to avoid cellular damage caused by crystallisation during refrigeration. On the day of the test, a portion of intestine was cut up into 1 cm² pieces, then quickly placed in a buffered solution at 4° C. for 15 min, then washed three times in buffer for 3×10 min, in order to eliminate excessive glycerol. The intestinal segments were thus preserved in the buffer 2 for a few minutes before they were used.

The enterocytes were prepared from washed intestinal pieces. The duodenal mucous membrane (microvilli) was carefully scraped at the surface with a clean glass microscope slide, then re-suspended in 2 ml of buffer. The enterocyte cells were dissociated from one another, and both the glycerol traces and the damaged or fragmented cells were eliminated by a series of washes and centrifugations at 3,000 rpm. Between each washing stage, a sample was observed using the Gram staining method, until the cells appeared isolated and in a perfect state. A well-prepared enterocyte solution may be preserved for 4 to 5 days at 4° C., without any risk of contamination.

In a 25 ml Erlenmeyer flask a 2 cm² intestinal piece was placed in 18 ml of buffer 1, to which 2 ml of lactic acid bacteria suspension were added.

The mixture was stirred gently, at 10 rotations per minute, at 37° C. for 30 min. There was no adherence to the enterocytes while the buffered solution remained cloudy, owing to the presence of bacteria in suspension. There was adherence to the enterocytes when the buffered solution once more became clear. Adherence could be confirmed using the Gram staining method, by bringing 500 μl of the lactic acid bacteria suspension, diluted 1/10 (10⁸ bacteria/ml), into contact with 500 μl of the previously prepared enterocyte suspension, in a hemolysis tube having a capacity of 3 ml.

In the event of non-adherence to the enterocytes, the purple lactic acid bacteria were anarchically dispersed around the (roseate) enterocytes.

In the event of adherence, the bacteria were conjoined, partially or entirely, to the enterocytes.

The lactobacilli to be retained were those for which adherence could be displayed.

Formulation of the glycerolised buffer (buffer 2):

1 part by volume of pure glycerol,

1 part by volume of Tris-EDTA buffer,

the composition of the Tris-EDTA buffer, which has a pH of 7.6, being as follows:

| | |
|---|---|
| tris hydroxymethylaminomethane: | 10 mM |
| ethylene diamine tetraacetic acid: | 5 mM |
| NaCl: | 0.155 M |
| water: | sufficient for 1 litre. |

Test Procedure for Displaying Autoaggregation and Coaggregation Properties (Properties b) and c), i.e. Adherence Between the Lactic Acid Bacteria of a Same Strain, or Between the Lactic Acid Bacteria of One Strain and the Bacteria of a Pathogenic Escherichia coli Strain.

The lactic acid bacteria were cultivated in a jar on MRS agar (Difco) at 37° C., in order to recreate microaerophilic conditions. The buffer 3, prepared from the following enzymatic mixture, was added to the MRS agar:

| | |
|---|---|
| Porcine gastric pepsin | 2 g |
| Porcine gastric mucus | 1 g |
| Porcine pancreatin | 1 g |
| Porcine bile extract | 4.5 g |
| NaCl solution at 5 g/l | 1 litre, |

by carrying out the following stages:

The pepsin and the NaCl aqueous solution were combined, and the pH was adjusted to 2 by adding 10 M HCl aqueous solution. The rest of the enzymes (mucus, pancreatin and bile extract) were added to this mixture in two stages. Meanwhile, the pH of an intermediate solution was set at 5 using a carbonate buffer 4 (30 ml of Na₂CO₃:0.2 M; 20 ml of NaHCO₃:0.2 M; and 200 ml of water) in order to reduce the risks of enzymatic denaturation. The pH of the final solution was then adjusted to 6 using the same buffer 4, described above.

The volume of the solution was then adjusted to 1 litre, with the NaCl solution at 5 g/l in a volumetric flask, and the pH was checked again to confirm a pH value of 6.

The Escherichia coli strains were cultivated for 18 hours at 37° C., on BHI agar in aerobiosis.

The BHI (brain heart infusion) agar was prepared by dissolving 52 g of the following mixture B in 1 litre of water. This agar has a pH of 7.4±0.2 at 25° C.

Mixture B

| | |
|---|---|
| Cattle brain infusion | 200 g |
| Cattle heart infusion | 250 g |
| Peptones | 10 g |
| Dextrose | 2 g |
| Sodium chloride | 5 g |
| Disodium phosphate | 2.5 g |
| Agar | 15 g |

The Escherichia coli and lactic acid bacteria colonies were withdrawn, using a sterile inoculating loop, at the surface of the agar, and were disaggregated in a volume of 2 ml of NaCl solution at 5 g/l, in order to form a homogeneous suspension. These suspensions were then diluted in the same buffer, in order to attain an approximate absorbance value of 1.5±0.1, measured at a wavelength of 600 nm.

A volume of 1 ml of lactic acid bacteria suspension was mixed with 2 ml of the buffer 3, described above. The same dilution was produced with the Escherichia coli suspension. The absorbences were thus adjusted to a value close to 0.5 (10⁸ bacteria/ml) for a wavelength of 600 nm. The 3 ml of lactic acid bacteria suspension were then added to the 3 ml of pathogenic bacteria suspension, in a single, sterile and hermetically sealed tube. After the tube had been turned a plurality of times over a period of 10 sec, a 1 ml sample was taken, in order to measure the absorbance (time: T0 hours). A further sample was then taken after 4 hours of incubation at 37° C., with mild stirring at 30 rpm (on a vertical rotational stirring table), for a last absorbance measurement (time: T4 hours). In the same incubation conditions, the controls required to measure the phenomenon of adherence between the bacteria of a same strain were carried out in the same manner, except that the 3 ml of lactic acid bacteria suspension were diluted in 3 ml of the buffer 3, described above. The concentrations of the various components of the enzymatic buffer during the aggregation tests were reduced (owing to the addition of the bacterial suspensions) and thus adjusted to desired values, which are: 1.3 g/l of pepsin, 0.6 g/l of gastric mucus, 0.6 g/l of pancreatin and 0.3% of bile extract.

The intensity of the phenomenon of adherence between bacteria (auto-aggregation) can be seen from the fact that the absorbance values of the negative controls at T0 hours and T4 hours only contained the bacteria of a lactobacillus or pathogenic strain, in suspension in a physiological solution.

% of auto-aggregation after 4 hours between bacteria of a same strain:

$$O . D . LC ( T0 hours ) - \frac{O . D . Lc ( T4 hours ) \times 100}{O . D . Lc ( T0 hours )} = \begin{matrix}\% of \\ auto \text{-} aggregation\end{matrix}$$

O.D. Lc(T4 hours)=absorbance of the lactobacilli suspension, measured after 4 hours

O.D. Lc(T0 hours)=absorbance of the lactobacilli suspension, measured at T0.

The percentage of co-aggregation was calculated using the standard equation of Handley et al. (Journal of General Microbiology, 1987, 133:3207-3217):

$$\frac{( O . D . Lc + O . D . Ec ) / 2 - ( O . D . L + O . D . E )}{( O . D . Lc + O . D . Ec ) / 2} \times 100 = \% co \text{-} aggregation$$

O.D. Lc and O.D. Ec correspond to the respective absorbance values, read at 600 nm, after 0 or 4 hours of incubation of the controls characterised solely by the lactobacillus strain or the E coli strain.

O.D. L+O.D. E corresponds to the absorbance value of the mixed cultures (lactobacilli+E coli), read after the same incubation period.

(O.D. Lc+O.D. Ec)/2 represents the average absorbance value of the mixed suspensions.

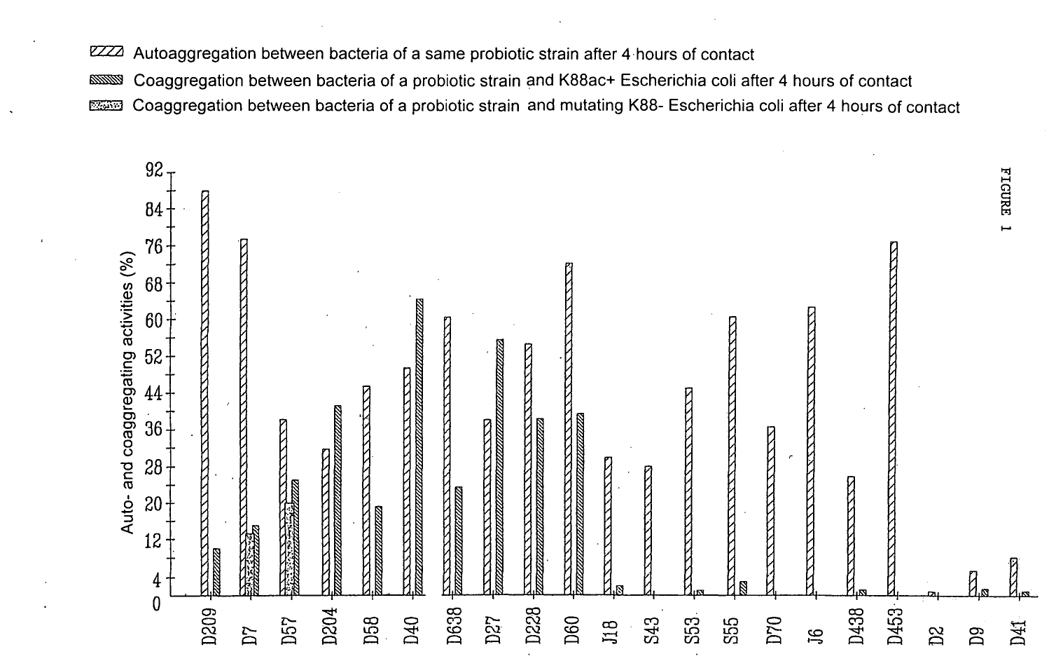

FIG. 1 shows the results obtained with regard to the properties of auto-aggregation and co-aggregation, in the case of 21 lactobacteria strains adhering to the porcine duodenal mucous membrane.

The results clearly show that the three selection criteria which were adopted (adherence, auto-aggregation and co-aggregation) could be observed simultaneously and are governed by recognition mechanisms that are in all likelihood different. Of the 70 strains adhering to the duodenal mucous membrane, only 10 were both auto- and co-aggregating. Certain strains may, however, be highly auto-aggregating, while at the same time developing a very low-intensity co-aggregation with E coli (strain J18, S55, S53, D438 of FIG. 1) and vice versa (strains D204, D40, D27 of FIG. 1).

The intensity of the phenomenon of co-aggregation may be greater than that of the phenomenon of auto-aggregation, even if the phenomenon of auto-aggregation has already attained a satisfactory level.

On the other hand, numerous probiotic strains adhering to the duodenal mucous membrane have not developed aggregating activity (e.g. strains D2, D9 and D41 of FIG. 1).

Test Procedure for Measuring the Specificity of the Phenomenon of Adherence to Pathogenic Bacteria Expressing K88 Fimbriae

For this study, the pathogenic bacteria strain used was a mutated Escherichia coli strain which no longer had the ability to express K88 fimbriae, but only type 1 fimbriae (common fimbriae encountered on the surface of all commensal, non-pathogenic E coli bacteria). The absence or the presence of K88 fibriae was confirmed after culture by carrying out a Fimbrex test (K88 Fimbrex kit, CVL, Weybridge).

This strain was cultivated on BHI agar at 37° C., the BHI agar being as defined above.

The properties of auto-aggregation and co-aggregation were displayed from the 10 previously selected lactobacilli strains, which, in the previous test (FIG. 1), had coaggregation values greater than or equal to 10.

The obtained results are also shown in FIG. 1.

Of the 10 adherent, auto-aggregating and coaggregating strains, only 2 lactobacilli strains developed a persistent coaggregating activity towards the mutated Escherichia coli strain, despite the absence of K88 fimbriae, while the other lactic acid bacteria strains did not have any property of coaggregating with said mutated strain.

A limited number of strains thus selected were able to adhere to the duodenal mucous membrane, to auto-aggregate and also to coaggregate specifically the K88 lectins of the enteropathogenic pathogenic species Escherichia coli (8 of the 70 that were initially isolated). The bacteria selected to produce medicinal preparations were represented by three Lactobacillus fermentum strains, 2 Lactobacillus acidophilus strains, 1 Lactobacillus helveticus strain and 1 Leuconostoc lactis strain.

1. Method of selecting non-pathogenic Gram-positive lactic acid bacteria strains that can combat infections by pathogenic bacteria for which the pathogenicity is the result of tissue adhesion, consisting in selecting a strain presenting all of the following properties:
a—the ability to adhere to tissues of an organ that is likely to be infected;
b—the ability of the bacteria of said strain to fix to one another via mutual adhesion sites; and
c—the ability to fix to the attachment determinants of the pathogenic bacterial strain responsible for the infection.

2. Method according to claim 1, characterised in that the Gram-positive lactic acid bacteria are selected from the bacteria that are naturally present in the infected organ of the host.

3. Method according to claim 1, characterised in that the lactic acid bacteria which fix in a selective manner to the attachment determinants of the pathogenic bacterial strain are selected.

4. Method according to claim 1, characterised in that the pathogenic bacterial strains belong to an enteropathogenic strain.

5. Method according to claim 4, characterised in that the pathogenic bacterial strain is a strain of the species Escherichia coli.

6. Method according to claim 1, characterised in that the infected organ used in the selection is a portion of the digestive tract.

7. Method according to claim 6, characterised in that the infected organ is the duodenum.

8. Therapeutic composition comprising a Gram-positive lactic acid bacteria strain selected by a method according to claim 1.

9. Use of a Gram-positive lactic acid bacteria strain, selected by the method according to claim 1, to produce a therapeutic composition that is intended to prevent or treat pathological conditions associated with an infection of a host organ by pathogenic bacterial strains.

10. Use according to claim 9, characterised in that the host is an animal.

11. Use according to claim 10, characterised in that the animal is a pig.

12. Method of producing a therapeutic composition, comprising the selection of a Gram-positive lactic acid bacteria strain by a method according to claim 1 and the mixing of the strain with a pharmaceutically acceptable vehicle.

13. Method according to claim 1, characterised in that properties b) and c) are assessed simultaneously in a buffered physiological medium reflecting the physiological conditions of the infected organ.

14. Method according to claim 13, characterised in that the infected organ is the duodenum, and in that the buffered physiological medium comprises an extract of duodenal mucus, the enzymes of digestive secretions and more particularly of bile salts, gastric secretions and pancreatic digestive secretions, and also the electrolytes that are naturally present in the duodenum and influence the pH.
